(19) United States
(12) Patent Application Publication
(10) Pub. No.: US 2011/0282178 A1
(43) Pub. Date: Nov. 17, 2011

(54) ADHESIVE STRAP FOR FOETAL MONITORING TRANSDUCERS AND THE LIKE

(76) Inventor: Betina Korshoj Andersen, London (GB)

(21) Appl. No.: 13/145,396

(22) PCT Filed: Jan. 14, 2010
(86) PCT No.: PCT/DK2010/000005

§ 371 Date: Jul. 20, 2011

(30) Foreign Application Priority Data
Jan. 21, 2009 (GB) 0900951.5

(51) Int. Cl. A61B 5/04; A61B 5/0444

(52) U.S. Cl. 600/391; 600/511; 600/588

(57) ABSTRACT

The invention relates to a system (10) for attaching to the stomach (24) of a pregnant woman, said system comprising a bracket (11) and strapping means; wherein said bracket comprising strapping engaging means (16) for releasable engaging said strapping means with said bracket (11) and/or for adjusting the effective length of said strapping means; wherein said bracket (11) either comprising a monitoring device (12); or comprising means for accommodating a separate monitoring device (12); wherein said strapping means comprise two separate straps (18) each having two ends; wherein one end of each of said separate straps (18) being an adhesive end (20) for adhering onto the skin of said woman's stomach (24).

### FIELD OF THE INVENTION

The present invention relates to foetal monitoring devices and like devices that have to be held to the body to co-act therewith e.g. for monitoring a foetal condition or contraction patterns during pregnancy and child birth.

In a first aspect the present invention relates to a system for attaching to the stomach of a pregnant woman, said system comprising a bracket and strapping means. In a second aspect, the present invention relates to a kit-of-parts comprising a system for attaching to the stomach of a pregnant woman and adhesive means. In a third aspect, the present invention relates to an apparatus for monitoring a foetus in the uterus of a pregnant woman. In a fourth aspect the present invention relates to the use of a system according to the invention or a kit-of-parts according the invention, or an apparatus according to the invention for monitoring the condition of pregnancy or child birth labour of a pregnant woman. In a fifth aspect the present invention relates to a method of setting up a system according to the invention, wherein the system comprising a monitoring device is placed on the skin of the stomach of the pregnant woman adjacent the foetus. In a sixth aspect the present invention relates to a method for monitoring the condition of pregnancy or child birth labour of a pregnant woman.

### TECHNICAL BACKGROUND

The majority of monitoring devices known from the prior art are held to the body with a strap. The transducer has loops or other strap-engaging means and the strap goes around the body to hold the transducer in position against the body.

Such prior art systems in which one strap is to be wound around the stomach of the woman are quite uncomfortable to wear to the pregnant woman. Furthermore, such prior art systems do not allow for immediate attachment of the monitoring device, i.e. within very few seconds in case of emergency situation due to complication of the condition of the pregnant woman or the foetus. Also, such prior art can be difficult to use when the woman is immobile on a bed due to the need for the strap to go around the body. When a woman is upright and moving, such prior art can pose problems in that the transducer signal can be difficult to maintain, due to the strap's inability to hold the required tension for an optimal signal. The prior art can also present problems when being used on large women as the straps come in standard sizes and rarely cater for very large women. Furthermore, the prior art poses a hygiene risk in that the straps are usually not cleaned between patients.

Hence, there exists a need for an improved system of monitoring devices which overcomes the above-identified drawbacks.

### BRIEF DESCRIPTION OF THE INVENTION

Such a system is provided according to a first aspect of the present invention.

Accordingly, in a first aspect the present invention relates to a system 10 for attaching to the stomach 24 of a pregnant woman, said system comprising a bracket 11 and strapping means;

wherein said bracket comprising strapping engaging means 16 for releasable engaging said strapping means with said bracket 11 and/or for adjusting the effective length of said strapping means;

wherein said bracket 11 either comprising a monitoring device 12; or comprising means for accommodating a separate monitoring device 12;

wherein said strapping means comprise two separate straps 18 each having two ends;

wherein one end of each of said separate straps 18 being an adhesive end 20 for adhering onto the skin of said woman's stomach 24.

In a second aspect the present invention relates to a kit-of-parts comprising a system for attaching to the stomach 24 of a pregnant woman and adhesive means, wherein said system comprising a bracket 11 and strapping means;

wherein said bracket 11 comprising strapping engaging means 16 for releasable engaging said strapping means with said bracket 11;

wherein said bracket 11 comprising means for accommodating a foetal monitoring device 12;

wherein said strapping means comprise two separate straps 18 each having two ends;

wherein said adhesive means is of a type capable of adhering one end 20 of each of said separate straps to the human skin.

In a third aspect the present invention relates to an apparatus for monitoring a foetus in the uterus of a pregnant woman comprising a system 10 according to the first aspect of the present invention or a kit-of-parts according to the second aspect of the present invention; wherein

said apparatus comprises a control unit for controlling said monitoring device of said system; and wherein

said apparatus comprises a data processing unit for processing the data received from said monitoring device; and wherein

said apparatus comprises a monitor or a printer for visual monitoring, such as real time visual monitoring, and/or a data logging device for logging data with the view to post measurement analysis, respectively.

In a fourth aspect the present invention relates to a use of a system according to the first aspect of the invention or of a kit-of-parts according to the second aspect of the invention, or an apparatus according to the third aspect of the invention for monitoring the condition of pregnancy or child birth labour of a pregnant woman.

In a fifth aspect, the present invention relates to a method of setting up a system 10 according to the first aspect of the invention, wherein the system comprising a monitoring device 12 is placed on the skin of the stomach 24 of the pregnant woman adjacent the foetus; and wherein the strapping means, comprising two separate straps 18 to engage the monitoring device 12 each having adhesive ends 20, is adhered at these ends to the body 14 to hold the monitoring device 12 in place.

In a sixth aspect the present invention relates to a method for monitoring the condition of pregnancy or child birth labour of a pregnant woman, said method comprising the steps according to the method according to the fifth aspect of the present invention and additionally comprises the step of monitoring said condition by means of said monitoring device and/or by means of an apparatus according the third aspect of the present invention.

The system of the first aspect of the present invention provides for ease of use when a woman is immobile and/or when being used on very large women, this being due to the fact that the system does not require circumferal attachment. Furthermore, the system allows for almost immediate attachment of the monitoring device, i.e. within very few seconds. Such swift attachment is of paramount importance in case of complication of the condition of the pregnant woman or the foetus. Compared to the prior art systems, in which the system comprises only one strap to be wound around the stomach of the woman, the system of the present invention according to a first aspect provides more comfort to the pregnant woman wearing the system. The system of the present invention also allows the woman to be upright and moving during monitoring without losing transducer signal strength. Further advantages include the ability to be repositioned and reused, this being due to the adhesive on the adhesive ends being repositionable, washable and reusable without losing its adhesive qualities. It also allows for cleaning before or after each patient use thus minimising hospital infections. The system of the present invention has the ability to be adapted to be used with all current transducer types.

### DESCRIPTION OF THE FIGURES

FIG. 1 is a partial side view of a pregnant woman's body, showing two monitoring devices embodying the invention positioned for foetal monitoring.

FIG. 2a is a plan view of one embodiment of the system according to the first aspect of the present invention.

FIG. 2b is a perspective view of the embodiment shown in FIG. 2a.

FIG. 2c is a side view of the embodiment shown in FIG. 2c.

FIG. 3a is a perspective view of an embodiment of the system according to the first aspect of the present invention in which the monitoring device 12 has been detached from the bracket 11.

FIG. 3b is a side view of the embodiment shown in FIG. 3a.

FIG. 4a is a perspective view of an embodiment of the system according to the first aspect of the present invention.

FIG. 4b is a side view of the embodiment shown in FIG. 4a.

FIG. 5a is a perspective view of an embodiment of the system according to the first aspect of the present invention.

FIG. 5b is a side view of the embodiment shown in FIG. 5a.

FIG. 6 is a plan view of an embodiment of the system according to the first aspect of the present invention.

FIG. 7 is a plan view of an embodiment of the system according to the first aspect of the present invention.

FIG. 8 is a plan view of an embodiment of the system according to the first aspect of the present invention.

FIG. 9 is a plan view of an embodiment of the system according to the first aspect of the present invention.

FIG. 10 is a perspective view of an embodiment of the system according to the first aspect of the present invention in which one of the straps 18 is yet to be attached to the monitoring device.

FIG. 11 is a perspective view of an embodiment of the system according to the first aspect of the present invention.

FIG. 12 is a perspective view of one of the straps 18 of the system according to the first aspect of the present invention.

FIG. 13 is a perspective view of an embodiment of the system according to the first aspect of the present invention.

FIG. 14a is a perspective view of an embodiment of the system according to the first aspect of the present invention.

FIG. 14b is a side view of the inter-engagable male and female parts 42 of FIG. 14a.

### DETAILED DESCRIPTION OF THE INVENTION

### The System According to the First Aspect of the Present Invention

In a first aspect the present invention relates to a system 10 for attaching to the stomach 24 of a pregnant woman, said system comprising a bracket 11 and strapping means;

wherein said bracket comprising strapping engaging means 16 for releasable engaging said strapping means with said bracket 11 and/or for adjusting the effective length of said strapping means;

wherein said bracket 11 either comprising a monitoring device 12; or comprising means for accommodating a separate monitoring device 12;

wherein said strapping means comprise two separate straps 18 each having two ends;

wherein one end of each of said separate straps 18 being an adhesive end 20 for adhering onto the skin of said woman's stomach 24.

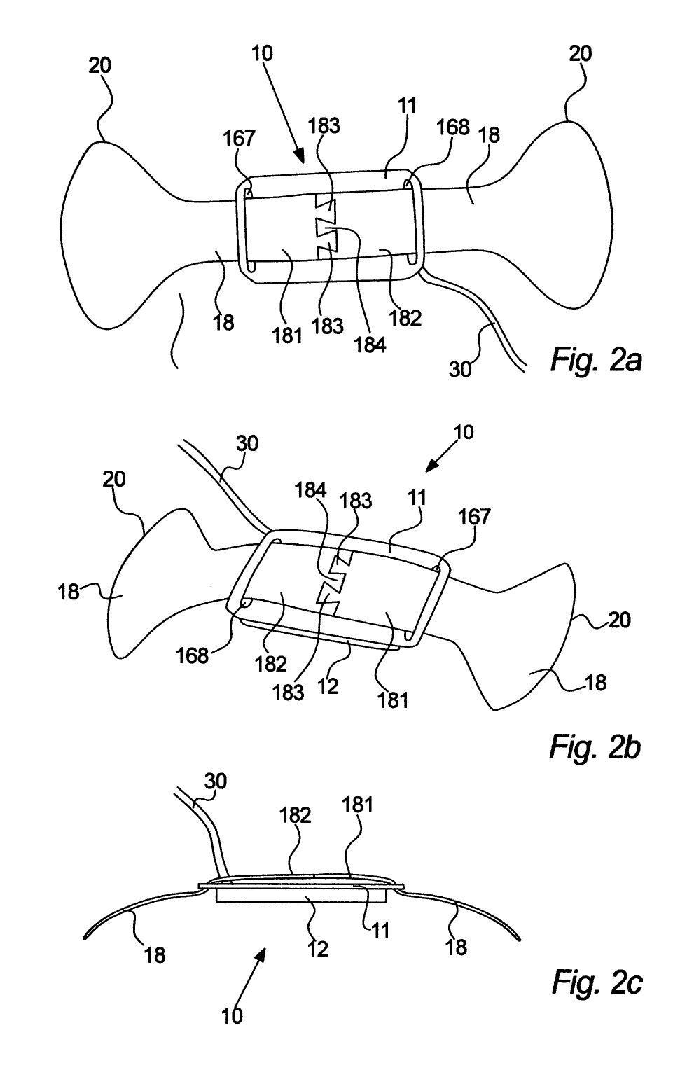

In a preferred embodiment of the system according to a first aspect of the present invention, the strapping engaging means 16 for releasable engaging said strapping means with said bracket 11 comprise loops 167, 168 on different sides of said bracket, such as on opposite sides of said bracket. Such an embodiment provides for easy and safe attachment of the straps 18 to the bracket 11. This embodiment is depicted in FIG. 2a, 2b, 2c, 3a, 3b, 7, 10, 14a.

In another preferred the strapping engaging means for releasable engaging said strapping means with said bracket 11 comprise mutual interlocking means at the distal end of each of said separate straps 18 relative to the adhesive end 20. Such an embodiment provides for easy and safe attachment of the straps 18 to the bracket 11. This embodiment is depicted in FIG. 2a, 2b, 2c, 3a, 3b, 7, 8, 9, 14a, 14b.

In a specific variation of this embodiment the mutual interlocking means comprises one or more wedged fingers 183,184 at the respective ends of said separate straps 18, wherein said wedged fingers 183 of one separate strap 18 is interlocking engageable with said wedged fingers 184 of said other strap 18. This embodiment is depicted in FIG. 2a, 2b, 2c, 3a, 3b.

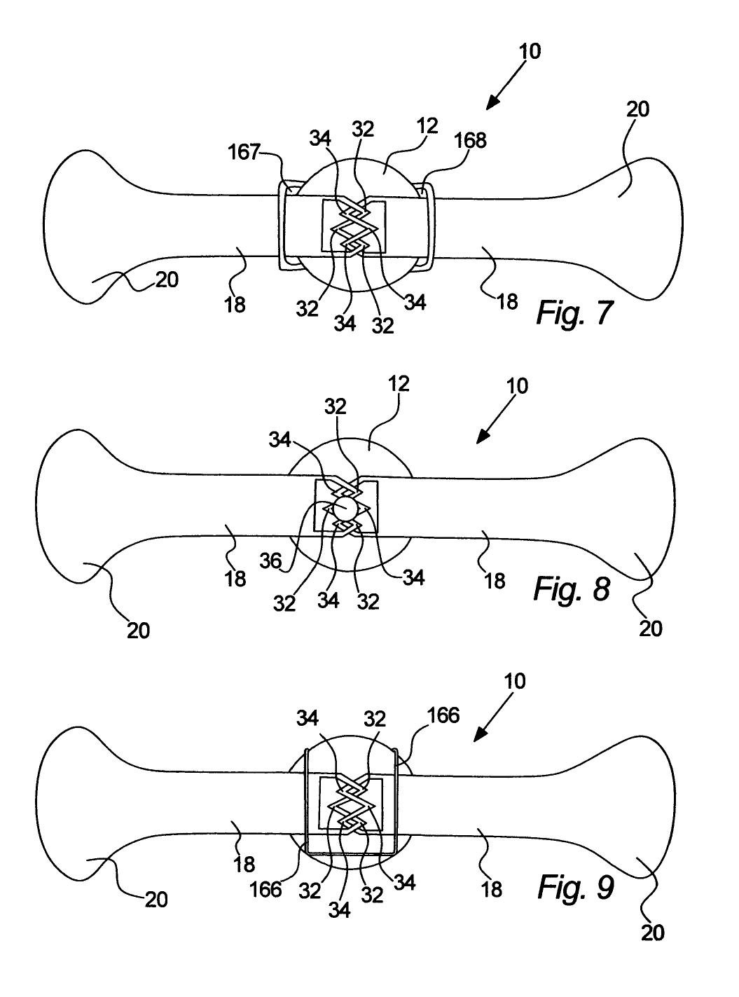

In another specific variation of that embodiment the mutual interlocking means comprises one or more inter-engageable apexes 32,34, such as three apexes, at the respective ends of said separate straps 18, wherein said apexes 32 of one separate strap 18 is interlocking engageable with said apexes 34 of said other strap 18. This embodiment is depicted in FIG. 7, 8, 9.

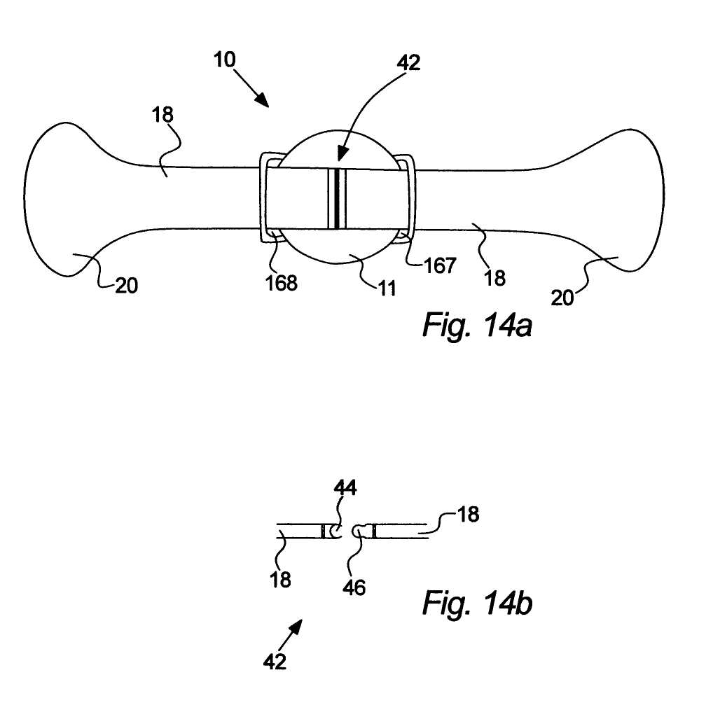

In yet another specific variation of that embodiment the mutual interlocking means comprises inter-engagable male and female parts 42 arranged at the respective ends of said separate straps 18, wherein said male part 46 of one separate strap 18 is interlocking engageable with said female part 44 of said other strap 18. This embodiment is depicted in FIG. 14a, 14b

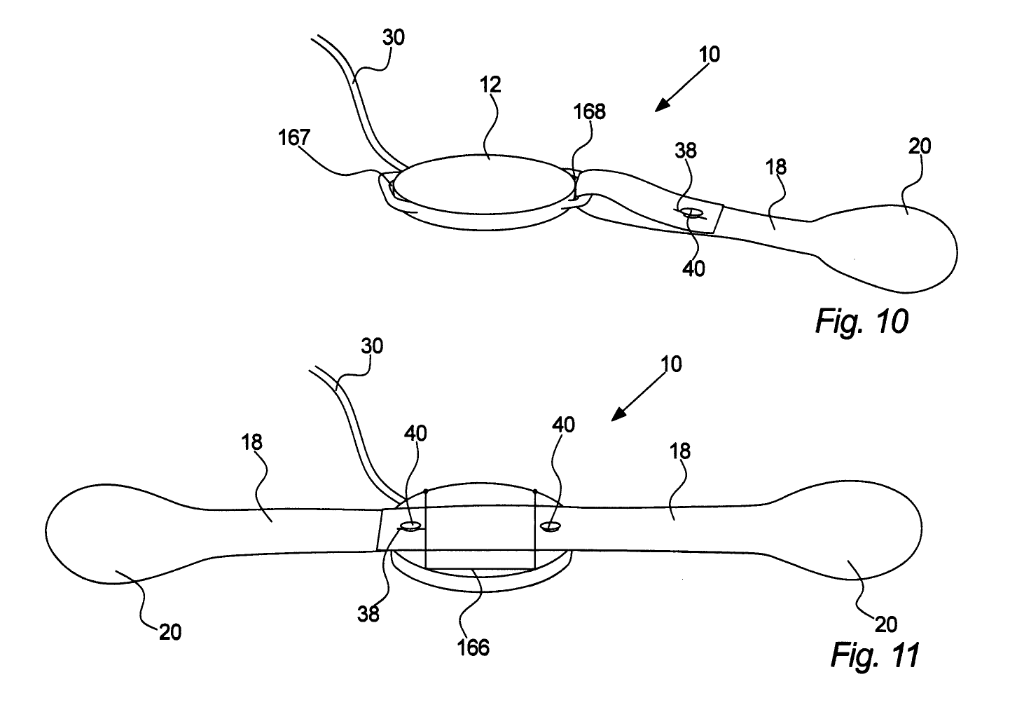

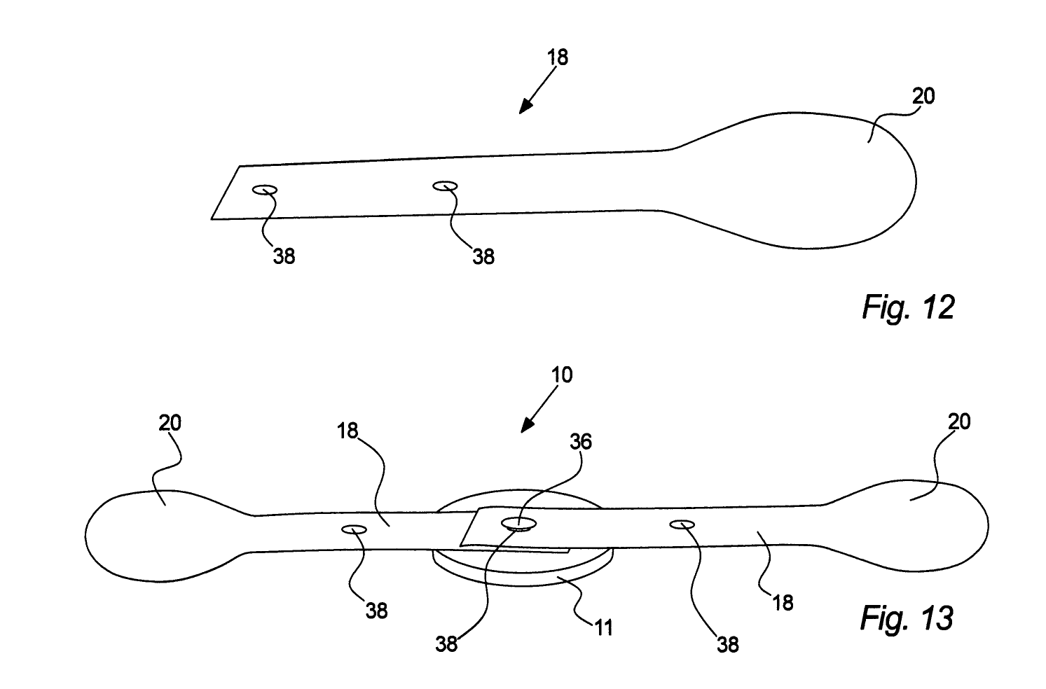

In another embodiment of the system of the first aspect of the present invention, the strapping engaging means 16 for releasable engaging said strapping means with said bracket 11 comprise one or more press studs 40 arranged on said bracket 11 and/or on said separate straps 18; or wherein the strapping engaging means for releasable engaging said strapping means with said bracket 11 comprise one or more buttons 36 arranged on said bracket, said buttons being engageable with said strapping means, such as by penetration of said buttons in hole(s) 38 arranged in said separate strap 18. This embodiment is depicted in FIGS. 10 and 11.

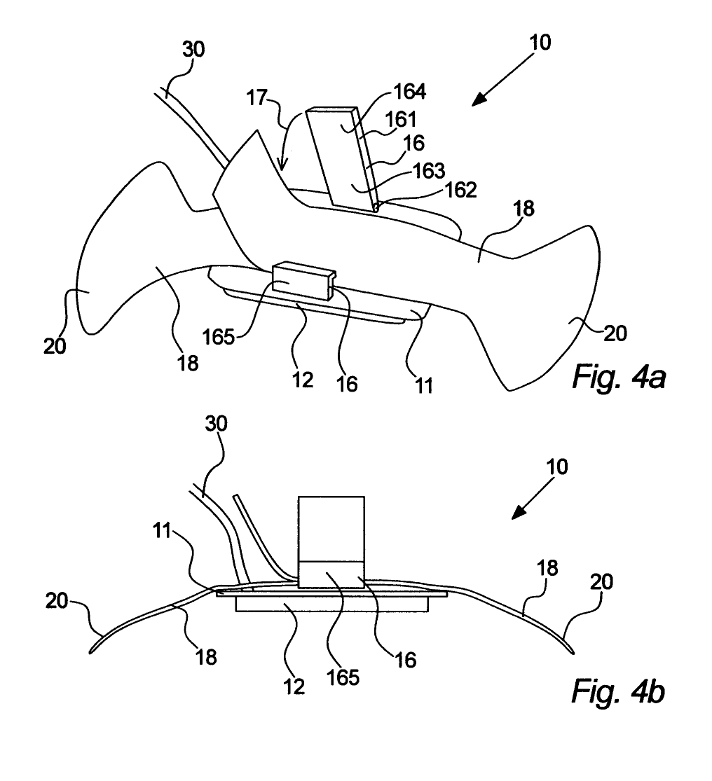

In yet another embodiment of the system of the first aspect of the present invention the strapping engaging means 16 for releasable engaging said strapping means with said bracket 11 comprise an arm 161 hinged by a hinge 162 and a catch 165 for locking said arm into a locked position. This embodiment is depicted in FIG. 4a, 4b, 5a, 5b.

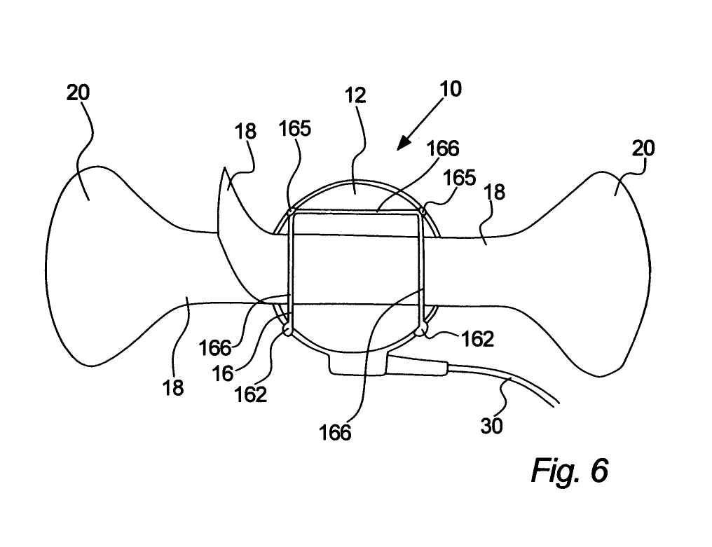

In still another embodiment of the system of the first aspect of the present invention the strapping engaging means 16 for releasable engaging said strapping means with said bracket comprise a wire loop 166 hinged by a hinge 162, and optionally spring means for locking said wire loop into a locked position. This embodiment is depicted in FIG. 6, 9, 11.

All the above specific embodiments have the advantage that they allow for easy and secure attachment of the straps 18 to the bracket 11 holding or comprising the monitoring device 12.

In another embodiment the monitoring device 12 is integrated with said bracket 11. This has the advantage that the design of the system is more simple and easier to maintain under hygienic acceptable conditions.

In another embodiment the bracket 11 and/or said monitoring device 12 comprise means for releasable fixing said monitoring device 12 to said bracket 11. This embodiment allows for exchanging one monitoring device for another kind on the same bracket 11 using the same straps 18.

In another embodiment the monitoring device 12 is an electronic monitoring device. This is a most preferable embodiment as many such types of monitoring devises are know.

In one variation of this embodiment the monitoring device 12 comprises a transducer and/or a receiver.

In another variation of this embodiment, the monitoring device 12 is a foetal monitoring device for monitoring foetal heartbeats and/or wherein the monitoring device is a monitoring device for monitoring contraction patterns during childbirth labour and/or pregnancy.

In yet another variation of this embodiment the monitoring device 12 is a toco transducer and/or an ultrasound transducer.

The above variations relating to the type of monitoring device are highly preferred due to their effectiveness in monitoring foetal conditions and/or pregnancy/labour conditions.

In one embodiment the strapping means is of a plastic or elastomeric material.

In another embodiment the strapping means is of polyurethane, polypropylene, polyethylene or silicone.

In yet another embodiment the strapping means is of an elastic or non-elastic fabric.

In still another embodiment the strapping means is of a foam material and/or a foam-like material.

The above variation relating to type of material of the straps 18 have shown to beneficial due to their provision of comport to the wearer and they are furthermore relatively inexpensive and easy to manufacture.

In one embodiment the length of each of said separate straps 18 independently is 60 cm or less, such as 50 cm or less, e.g. 40 cm or less, for example 30 cm or less, such as 20 cm or less, such as 10 cm or less.

In one embodiment the width of each of said separate straps 18 independently is 2-9 cm, such as 3-8 cm, e.g. 4-7 cm, such as 5-6 cm.

In one embodiment the thickness of each of said separate straps 18 independently is 1-8 mm, such as 2-7 mm, for example 3-6 mm, such as 4-5 mm.

The above dimensions have proven to very comfortable to the wearer. Furthermore they will offer sufficient holding power provide for safe attachment of the bracket 11/device 12 to the body 14 of the pregnant woman.

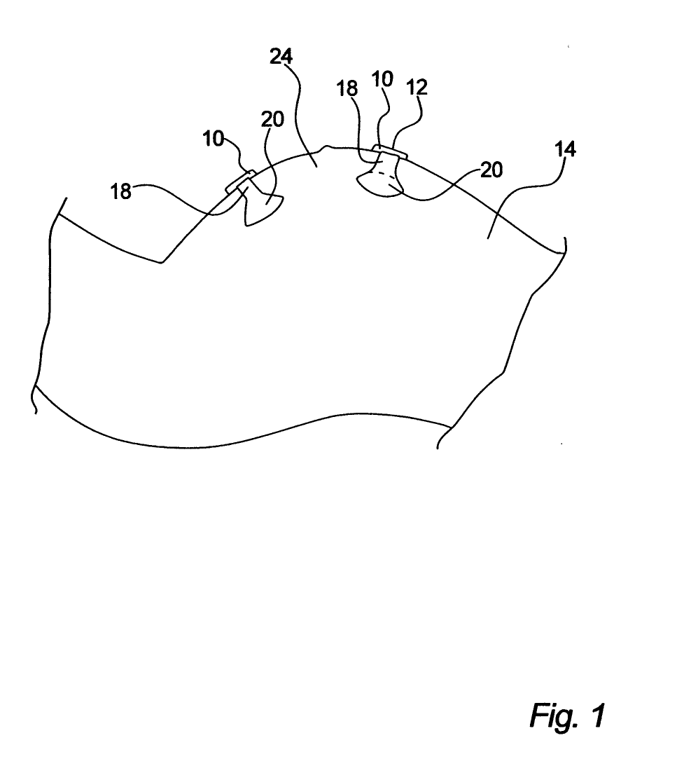

In a preferred embodiment the adhesive end 20 of each of said separate straps 18 is splayed. This has the advantage of providing improved holding power of the straps 18 to the body 14 of the pregnant woman. This embodiment is depicted in FIG. 1, 2a, 2b, 2c, 3a, 3b, 4a, 4b, 5a, 5b, 6, 7, 8, 9, 10, 11, 12, 13, 14a.

In a preferred embodiment of the system according to a first aspect of the present invention, the adhesive effect of said adhesive end 20 of said separate straps 18 is provided by a coating of adhesive 22.

In a variation of this embodiment the adhesive 22 is a pressure-sensitive adhesive.

In another variation of that embodiment the adhesive end 20 is provided with a silicone gel adhesive 22 or an acrylic gel adhesive 22.

The above embodiments relating to the adhesive have shown to provide the necessary strength to the system of the first aspect.

It is preferred that adhesive 22 being of a type which is washable in water without losing its adhesive effect in a detrimental way in respect of being reusable.

This embodiment provides for reusing the straps of the system on different persons without compromising hygienic concerns,

In one embodiment the adhesive 22 is hypoallergenic to the human skin and/or is antibacterial. It is contemplated that an antibacterial effect can be obtained by including in the adhesive metallic silver, such as grains of metallic silver.

The adhesive per se may be of the type PS-1270, from Polymer Science Inc. Also the adhesives from Scapa under the names Bioflex Rx1267P, Bioflex Rx1248P and Bioflex Rx1268S haven proven suitable.

Such an embodiment provides better hygienic and health compliance to the system.

In one embodiment of the system according to the first aspect of the present invention each of said two separate straps 18 comprising length adjusting means for adjusting the effective lengths of said straps.

In a variation of this embodiment, the length adjusting means are located at the distal end of each of said separate straps relative to the adhesive end 20.

In a special variation of that embodiment the length adjusting means comprises fasteners of the type hook-and-loop fasteners.

In another variation of that embodiment, the length adjusting means comprises buttonholes 38 on one or two of said separate straps, said buttonholes being engageable with a corresponding knob or button 36 on said bracket 11. This embodiment is depicted in FIG. 10, 11, 12, 13.

In another variation of that embodiment, length adjusting means comprises buttonholes 38 on one or two of said separate straps, said buttonholes being engageable with a corresponding separate knob or button for connecting said separate straps to each other, said separate knob or button being in the form of a double-headed rivet, said double-headed rivet comprising a stem comprising two heads arranged at opposite ends of said stem.

The embodiment relating to the length adjusting means all provides for better flexibility in relation to fitting the system to women in various stages of the pregnancy in that the system thereby allows for being fitted to various stomach sizes. Furthermore, such length adjusting means provides for better comport to the wearer.

In a preferred embodiment of the system according to the first aspect of the present invention the monitoring device 12 comprises a cable 30 for being connected to a control unit, or wherein said monitoring device comprises wireless means for being wirelessly connected to a control unit.

This embodiment provides for being able to monitoring in case the control unit is not integrated in the device per se. it is however contemplated that the control unit is integrated in the device 12 or in the bracket 11.

In a preferred embodiment, the system is a combination of a device 12 to co-act with the body, that has loop 167,168 or other strap-engaging means and at least two straps 18 that engage such means and has adhesive ends 20 to affix the body 14 to hold the device 12 to the body 14 to co-act therewith.

### The Kit-of Parts According to the Second Aspect of the Present Invention

In a second aspect, the present invention relates to a kit-of-parts comprising a system for attaching to the stomach 24 of a pregnant woman and adhesive means, wherein said system comprising a bracket 11 and strapping means;

wherein said bracket 11 comprising strapping engaging means 16 for releasable engaging said strapping means with said bracket 11;

wherein said bracket 11 comprising means for accommodating a foetal monitoring device 12;

wherein said strapping means comprise two separate straps 18 each having two ends;

wherein said adhesive means is of a type capable of adhering one end 20 of each of said separate straps to the human skin.

This aspect is advantageous in that it provides for the choice of applying different types of adhesives to the straps in order to obtain better patient compliance in the case, where the pregnant woman has sensitive skin towards one type of adhesive.

In one embodiment the system other than the adhesive is as defined above in respect of the first aspect of the present invention.

In one embodiment the adhesive is as defined above in respect of the first aspect of the present invention.

In one embodiment the adhesive means is a liquid adhesive 22 to be applied on the one end 20 of each of said straps 18.

In another embodiment the adhesive means is an adhesive sheet 22 to be applied on the one end of each of said straps 18.

### The Apparatus According to the Third Aspect of the Present Invention

In a third aspect, the present invention relates to an apparatus for monitoring a foetus in the uterus of a pregnant woman comprising a system 10 according to the first aspect of the present invention or a kit-of-parts according to the second aspect of the present invention, said apparatus comprises a control unit for controlling said monitoring device of said system; and wherein

said apparatus comprises a data processing unit for processing the data received from said monitoring device; and wherein

said apparatus comprises a monitor or a printer for visual monitoring, such as real time visual monitoring, and/or a data logging device for logging data with the view to post measurement analysis, respectively.

The apparatus is suitable in case the device 12 or the bracket 11 does not itself comprise monitoring and control means.

### The Use According to the Fourth Aspect of the Present Invention

In a fourth aspect the present invention relates to the use of a system according to the first aspect of the present invention or a kit-of-parts according to the second aspect of the present invention, or an apparatus according to the third aspect of the present invention for monitoring the condition of pregnancy or child birth labour of a pregnant woman.

In one embodiment, the use is for foetal and/or contraction monitoring of a pregnant woman.

In one embodiment more than one monitoring device is used.

In one embodiment these more than one monitoring devices comprise monitoring devices for monitoring different types of conditions.

These embodiments have the advantage that more than one parameter of the condition of the foetus can be monitored simultaneously.

### The Method According to the Fifth Aspect of the Present Invention

In a fifth aspect, the present invention relates to a method of setting up a system 10 according to the first aspect of the present invention, wherein the system comprising a monitoring device 12 is placed on the skin of the stomach 24 of the pregnant woman adjacent the foetus; and wherein the strapping means, comprising two separate straps 18 to engage the monitoring device 12 each having adhesive ends 20, is adhered at these ends to the body 14 to hold the monitoring device 12 in place.

In this method the monitoring device 12 is placed on the skin adjacent the foetus and straps to engage the device 12 or the bracket 11 accommodating the device and the adhesive ends are adhered at these ends to the body to hold the device/bracket in place. The strap may be engaged with the device/bracket before or after the strap is adhered to the body. For example, the straps may be threaded through loops of the device/bracket to engage the same and then adhered to the body. Again, the two ends of the straps may be adhered to the body and the device/bracket then engaged with the strap to be held to the body thereby, e.g. by placing the device under the straps (after the straps are adhered to the body) and then engaging the straps with the device/bracket by means of a clip on the back of the device/bracket, or by having the straps unfastened at its middle into two parts (before or after these are adhered to the body), then threading these two parts through respective loops on the device/bracket and then fastening these two parts together so that the straps hold the device to the body.

The straps may have these two parts threaded through respective loops on the device/bracket and then these two parts are fastened so that the straps when adhered to the body hold the device to the body. Preferably, the straps, unfastened at its middle, have these two straps threaded through respective loops on the device/bracket and then these two parts are fastened together so that the straps when adhered to the body holds the device to the body.

In one embodiment a sound-transmitting gel is applied to the skin prior to placing the monitoring device on the skin.

In another embodiment more than one monitoring device 12 are used.

In a variation of this embodiment said more than one monitoring devices 12 comprise monitoring devices 12 for monitoring different types of conditions.

In another variation of this embodiment said more than one monitoring devices 12 comprise monitoring devices 12 for monitoring foetus heartbeat and for monitoring contraction patterns during pregnancy or childbirth.

Preferably the monitoring device 12 is placed on the skin of the abdomen of the pregnant woman.

### The Method According to the Sixth Aspect of the Present Invention

In a sixth aspect, the present invention relates to a method for monitoring the condition of pregnancy or child birth labour of a pregnant woman, said method comprising the steps of the method according to the fifth aspect of the present invention and additionally comprises the step of monitoring said condition by means of said monitoring device and/or by means of an apparatus to the third aspect of the present invention.

Referring to the drawings, the present invention will now be described by reference to specific embodiments.

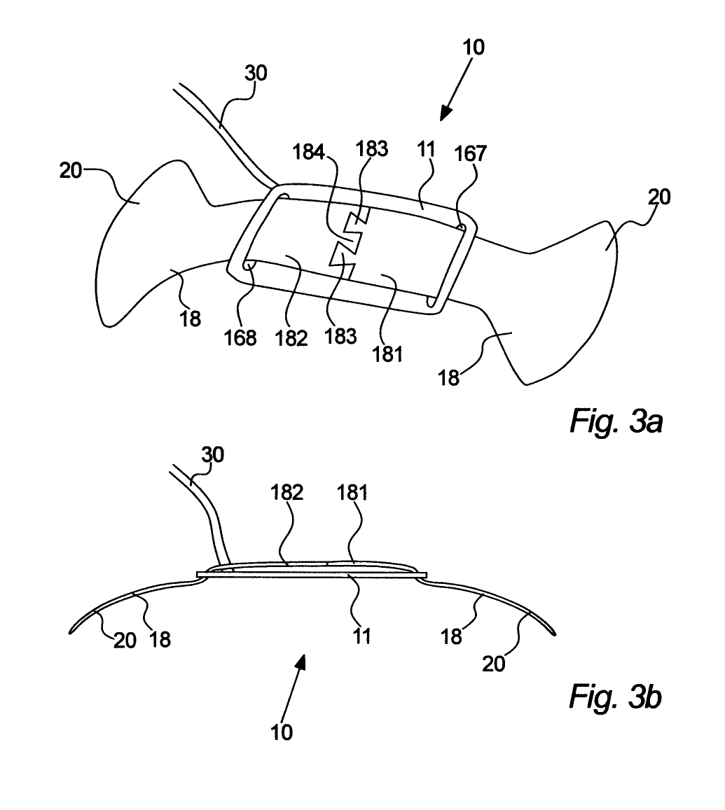

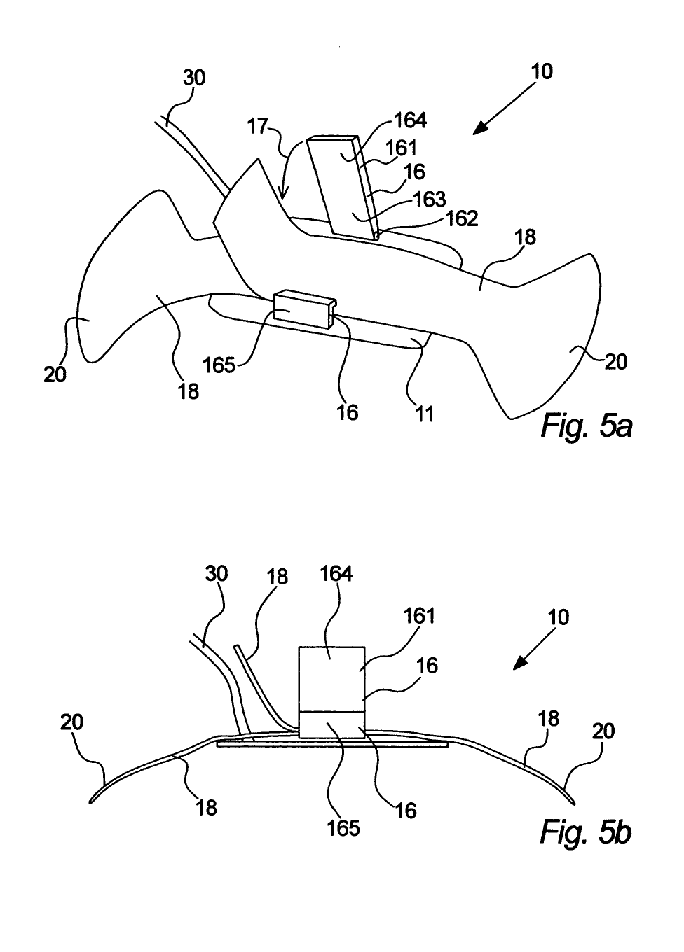

A system 10 comprises a device 12 to co-act with the body 14 that has strap-engaging means 16 and straps 18 that engage such means 16 and has adhesive ends 20 to affix to the body 14 to hold the device 12 to the body 14 to co-act therewith. The adhesive 22 of said ends is provided by the ends 20 being coated with an adhesive, such as a pressure sensitive adhesive 22. Said device 12 is exemplified as a foetal monitoring transducer 12 for monitoring the heartbeats of a foetus. It is placed on the stomach 24, usually with the interposition of a sound-transmitting gel, on the underside of transducer 12, and held in place by the straps 18, and is adjustable in position by being able to be slid along the straps 18. The straps 18 are means to apply tension to hold the transducer 12 firmly or tightly to the skin of body 14, sufficiently for good operation of the monitoring device 12. The strap-engaging means 16 of the system 10 comprises a loop 167,168 on either side of the bracket 11 in the embodiment of FIGS. 2a, 2b, 2c, 3a, 3b, 7 and 10. Means 16 in the FIGS. 4a and 4b embodiment comprises a clip 16 in the form of an arm 161 hinged by a hinge 162 at one end 163 of arm 161 to the back 121 of the monitoring device 12 with its other, free end 164 able to close (in the direction of arrow 17) over the straps 18 and clip to the device 12 by engaging under a catch 165 to engage the straps 18. Means 16 in the FIGS. 6, 9 and 11 embodiment comprises a clip 16, in the form of a (rectangular) loop of wire 166 hinged by hinges 162 at its ends 163, 164 to the device 12 so that the free end 166 of the loop 166 is able to close over the straps 18 and clip to the device 12 by spring action at the hinges 162 or by engaging under catch means 165 to engage the straps 18. Any such means 16 may allow the bracket 11 and/or the device 12 to be moved along the straps 18. As shown in FIGS. 1, 2a, 2b, 3a, 4a, 5a, 6, 7, 8, 9, 10,11, 12, 13, 14a, said ends 20 are splayed. In the embodiments shown in FIGS. 2a, 2b, 2c, 3a and 3b the straps 18 have a middle fastening 28 (in the form of a set 183,184 of (wedged) fingers on each part 181,182, which sets interdigitate to interengage to hold together parts 181,182 in a known manner of fastening) at which it can be separated into two parts 181,182 suitable to be inserted through respective suitable loops 167,168 on the device 12 or the bracket 11 and then fastened together over the bracket 11 or the device 12, so that the device 12 can then still slide along the straps 18. The straps 18 is able to be reused, due to the adhesive 22 being washable (e.g. like the adhesive used for a TENS machine pad) or cleanable, the adhesive may be a silicone adhesive and/or having a protective cover that can be peeled off to expose the adhesive.

In a method of setting up the foetal monitoring device 12, the device 12 is placed on the skin of the stomach 24 adjacent the foetus (not indicated) and the straps 18 to engage the transducer 12 or the bracket 11 are adhered at its ends 20 to the body 14 to hold the device 12 in place. The straps 18 may be engaged with the device 12 by any of the methods indicated above. For example, the straps 18, unfastened at its middle into two parts 181,182, has these two parts 181,182 threaded through respective loops 167,168 on the device 12/bracket 11 and then these two parts 181/182 are fastened together so that the straps 18 when adhered to the body 14 hold the device 12 to the body 14.

It should be noted that in the present description and the appended claims, the following definitions shall be adhered to:

“Strapping means” shall be interpreted as “straps”.

“Strapping engaging means” shall be interpreted as “engagers for strapping means”.

“Means for accommodating a separate monitoring device” shall be interpreted as “an accomodater for a separate monitoring device arranged on the bracket” and/or as “an accomodater for a bracket arranged on a separate monitoring device”.

“Mutual interlocking means” shall be interpreted as “mutual interlockers”.

“Spring means” shall be interpreted as a “spring”.

“Means for releasable fixing a monitoring device to a bracket” shall be interpreted as “releasable fixation elements for fixing a monitoring device to a bracket”.

“Length adjusting means for adjusting the effective lengths of straps” shall be interpreted as “length adjusters for adjusting the effective lengths of straps”.

“Wireless means for being wirelessly connected to a control unit” shall be interpreted as “wireless connectors”.

“Adhesive means” shall be interpreted as “adhesive”.

“Means for accommodating a foetal monitoring device” shall be interpreted as “an accomodater for a foetal monitoring device”.

It will be apparent to one skilled in the art that features of the different embodiments disclosed herein may be omitted, selected, combined or exchanged and the invention is considered to extend to any new and inventive combination thus formed. Where a preference or particularisation is stated, there is implied the possibility of its negative, i.e. a case in which that preference or particularisation is absent.

Many variations of the invention and embodiments hereinbefore described will be apparent to people skilled in the art and all such variations are to be considered as falling within the scope of the invention.

1-53. (canceled)

54. A system for attaching to the stomach of a pregnant woman, said system comprising a bracket and straps;
said bracket comprising an engager for releasably engaging said straps with said bracket and allowing said bracket to be moved along said straps and optionally for adjusting the effective length of said straps;
said bracket either comprising a monitoring device or comprising an accommodator for a separate monitoring device;
said straps comprising two separate straps each having two ends;
one end of each of said separate straps being an adhesive end for adhering onto the skin of said woman's stomach.

55. The system according to claim 54, wherein the engager for releasably engaging said straps with said bracket comprises loops on different sides of said bracket.

56. The system according to claim 55, wherein the engager for releasably engaging said straps with said bracket comprises a mutual interlocker at the further end of each of said separate straps.

57. The system according to claim 56, wherein said mutual interlocker is selected from the group consisting of:
one or more wedged fingers; one or more inter-engageable apexes, and inter-engageable male and female parts, said mutual interlocking means being disposed at the respective further ends of said separate straps.

58. The system according to claim 54, wherein the engager for releasably engaging said straps with said bracket comprises one or more press studs arranged on at lest one of said bracket and said separate straps.

59. The system according to claim 54 wherein the engager for releasably engaging said straps with said bracket comprises at least one button arranged on said bracket, said at least one button being engageable with said straps.

60. The system according to claim 54, wherein the engager for releasably engaging said straps with said bracket comprises an arm hinged by a hinge and a catch for locking said arm into a locked position

61. The system according to claim 54 wherein the engager comprises a wire loop hinged by a hinge.

62. The system according to claim 54, wherein said monitoring device is integrated with said bracket.

63. The system according to claim 54, wherein at least one of said bracket and said monitoring device comprises means for releasably fixing said monitoring device to said bracket.

64. The system according to claim 54, wherein the monitoring device is selected from the group consisting of:
a foetal monitoring device for monitoring foetal heartbeats;
a monitoring device for monitoring contraction patterns during childbirth labour or pregnancy; a toco transducer, and an ultrasound transducer.

65. The system according to claim 54, wherein the straps are composed of a material selected from the group consisting of: polyurethane, polypropylene, polyethylene and silicone.

66. The system according to claim 54, wherein said adhesive is hypoallergenic to the human skin.

67. The system according to claim 54 wherein said adhesive is antibacterial and contains metallic silver.

68. The system according to claim 54, wherein each of said two separate straps comprises length adjusters for adjusting the effective lengths of said straps.

69. The system according to claim 68, wherein said length adjusters are selected from the group consisting of: hook-and-loop fasteners, and buttonholes on at least one of said separate straps, said buttonholes being engageable with a corresponding projection on said bracket.

70. The apparatus for monitoring a foetus in the uterus of a pregnant woman comprising a system according to claim 54; wherein
said apparatus comprises a control unit for controlling said monitoring device of said system; and wherein
said apparatus comprises a data processing unit for processing the data received from said monitoring device.

71. The apparatus according to claim 70, further comprising a data logging device for post measurement analysis.

72. A kit of parts comprising a system for attaching to the stomach of a pregnant woman and adhesive means, wherein said system comprises:
a bracket and straps, said bracket comprising an engager for releasably engaging said straps with said bracket and for allowing said bracket to be moved along the straps;
said bracket comprising an accommodator for accommodating a foetal monitoring device;
wherein said straps comprise two separate straps each having two ends;
wherein said adhesive means is of a type capable of adhering one end of each of said separate straps to the human skin.

73. A method of monitoring a condition selected from the group consisting of pregnancy and child birth labour in a pregnant woman, the method comprising the steps of:
providing straps, said straps comprising two separate straps, each strap having an adhesive end provided with skin adhesive and a further end, said further ends being fastenable together when mutually adjacent;
attaching said adhesive ends to spaced-apart skin regions of the pregnant woman with said further ends mutually adjacent in a stomach region of the pregnant woman;
locating a monitoring device on said strapping means, said monitoring device being movable along said strapping means and fastening together said further ends to retain the monitoring device against the skin of the pregnant woman;
moving the monitoring device along the strapping means to a desired position, and
monitoring said condition with said monitoring device.

74. The method according to claim 73 wherein said desired position is adjacent a foetus, said monitoring device is a foetal monitoring device and a condition of said foetus is monitored.

75. The method according to claim 73 wherein said monitoring device is a device for monitoring contraction patterns during childbirth labour and contraction patterns are monitored.
